(19) United States
(12) Patent Application Publication
(10) Pub. No.: US 2009/0263523 A1
(43) Pub. Date: Oct. 22, 2009

(54) Enhancer of Cholinesterase Activity

(75) Inventors: Hideaki Matsuda, Osaka-fu (JP); Yoshimasa Miyake, Osaka-fu (JP); Noriko Hirata, Osaka-fu (JP)

Correspondence Address: WENDEROTH, LIND & PONACK, L.L.P., 1030 15th Street, N.W.,, Suite 400 East, Washington, DC, 20005-1503, US

(73) Assignee: Veritron Limited

(21) Appl. No.: 11/992,315

(22) PCT Filed: Sep. 21, 2006
(86) PCT No.: PCT/JP2006/319219

§ 371 Date: Mar. 20, 2008

(30) Foreign Application Priority Data
Sep. 22, 2005 (JP) 2005-276100

(51) Int. Cl. A61K 36/48

(52) U.S. Cl. 424/779

(57) ABSTRACT

The invention provides an enhancer for cholinesterase. An enhancer comprising Gam Arabic as an active ingredient is useful for preventing and/or treating a decreased cholinesterase activity due to liver damage or inhibition of cholinesterase as a side effect of various agents.

### TECHNICAL FIELD

This invention relates to an enhancer of cholinesterase activity comprising Gum Arabic (GA) as an active ingredient. Specifically, it relates to a medicine for preventing and/or treating a decreased cholinesterase activity due to liver damage or inhibition of cholinesterase as a side effect of various agents.

### BACKGROUND ART

Recently, use of a chemical pesticide is on the way to be controlled in the agriculture of our country but a large amount of pesticide is often found in a vegetable imported from a foreign country and it becomes a problem. It is known that a cholinesterase activity in blood and/or brain is inhibited for a long period and a cholinergic activity through muscarine receptor is decreased in CNS when an organophosphorous pesticide is taken in with foods [cf. non-patent, document 1].

Cholinesterase is a ubiquitous enzyme in a living organism and often inhibited by various agents, especially anti-cancer agent or organophosphorus compound, as well as a cholinesterase-inhibitor like neostigmin etc., and a decreased cholinesterase activity is one of the reasons causing side effects of various agents [patent document 1].

GA is a gummy exudation obtained from the stems and/or branch of Acacia senegal (Leguminosae) or more undetermined Acacia species (Leguminosae). Acacia senegal originally comes from a dry region between Senegal and Ethiopia in Africa, and is now widely distributed in India.

It is a small tree, about 6 m in height, and has a gray smooth bark and a branch with zigzag shape. It is characterized by three thorns attached to each leaf, wherein the central thorn is curved, and having cream colored flowers. GA is one of naturally collected resins, a colorless or, pale yellow transparent ball-like lump. Sometimes it has milky colored nontransparent appearance.

Most of commercially available GA used for industry is the ball-shaped gummy exudation collected from this species. Other gummy exudation collected from Acacia seyal is also commercially available.

The said gummy exudation obtained from the stems and/or branch of Acacia species has been used as paste since the pre-Christian era. It was called Gam Arabic since the said gummy exudation was produced in Africa and exported through Arabia to other foreign countries.

GA is a colorless or pale yellow transparent ball-like lump/pieces and sometimes has milky colored nontransparent appearance. It has many cracks on the surface, and is easily broken; when broken, the new surface is glassy and often sheds luster. GA is odorless, tasteless and viscous. The major component of GA is arabic acid(79 to 81%), which exists as Ca, Mg and/or K salts.

Acid hydrolysis of GA yields L-arabinose, D-galactose, L-rhamnose, and D-glucronic acid. In addition trace of hydrolase and oxydase are present, together with a little amount of mineral and proteins.

GA has been used as a binder for fixing a pigment from the past, and still used as a binder for transparent and nontransparent water paints at present. In addition to water paint, GA is widely used as an emulsifying agent in confectionary, such as candies or soft drinks, and as an additive when manufacturing a sugar-coated tablet or syrup in pharmaceuticals.

GA is a familiar material and a binder comprising about 35% of GA is also used as a medium for giving transparency to a water paint and/or gloss to a picture painted with the water paint, as well as used as a binder when manufacturing the water paint. 3% solution of GA is useful as a binder for a writing brush after washed out.

From the era of ancient Egypt, GA. has been used as a folk medicine for the treatment of periodontal disease and alveolar pyorrhea (treating a bleeding from the gums, removing ulcers in the gums, or promoting a growth of the teeth;), lung disorder and liver disorder.

At present, GA is used as an emulsifier with flavor for keeping homogeneity of ingredients of juices and ice-creams, a food additive for maintaining a shape of candies and stabilizers of medicines for compressing tablets or preventing disproportionation of the ingredients in a liquid formulation.

Although it has been reported that Gum Arabic have a decreasing effect on blood cholesterol and triglyceride level in human [Non-patent document 2], improving effect on intestinal flora. [Non-patent document 3] and protective effect on acetaminophen-induced hepatotoxicity. [Non-patent document 4], an efficacy of Gum Arabic as an enhancer of the effect of adrenocortical hormone agent in nephritis, and an efficacy of Gum Arabic as a reducer of the side effects of adrenocortical hormone agent have not been reported.

[Patent document 1]Tokkyo Kokai 7-228529(1995).

[Non-patent document 1]J. E. Gibson, R. K. D. Peteerson and B. A. Shurdut, Environ. Health Perspect. 106(1998),303-305; Toxic effect of pesticides: in C. D. Klaassen (Ed.), Casarett and Doull's Toxicology, The BasicScience of Poisons, McGrawHill, New York 1996, 643-689, D. J. Ecobichon

[Non-patent document 2]Ross A H, Eastwood M A, Brydon W G, Anderson J R, Anderson D M., Am J Clin Nutr., 37, 368-75, 1983.

[Non-patent document 3]Phillips, G. O., Food Addit. Contam. 15, 251-264, 1998.

[Non-patent document 4]Gamal el-din A M, Mostafa A M, Al-Shabanah O A, Al-Bekairi A M, Nagi M N., Pharmacological Research, 48, 631-635, 2003.

### DISCLOSURE OF INVENTION

The present invention was made in order to provide a medicine to inhibit a decrease of cholinesterase activity caused by a pesticide or other agent, or to enhance the cholinesterase activity without side effects.

The inventors focused on GA which has been utilized as a folk medicine since ancient times in Arabic and/or African area, and completed the present invention finding that GA inhibited the decrease of cholinesterase activity caused by Chlorpyrifos, one of organophosphorous pesticides.

Efficacy of GA to prevent and/or treat liver damage has been suggested in several reports, but these effects are explained by the antioxidative activity or inhibitory effect on absorption of liver damage-inducing substance, which are already known as to GA.

As shown in the following example, a decrease of cholinesterase activity was inhibited but an increase of LDH etc. was not significantly changed when GA and Chlorpyrifos were administered simultaneously.

This means that AG specifically inhibits a decrease of cholinesterase activity caused by Chlorpyrifos and does not inhibit absorption of Chlorpyrifos. Thus, the invention is useful for specifically controlling a cholinesterase activity when preventing and/or treating a liver damage.

As the water-soluble gum in this invention, a gummy exudation from the stems and/or branch of Acacia species (Leguminosae) such as Acacia senegal and Acacia seyal can be used as its intact form. From the view- point of easy availability and easy formulation, the dried powder of the said gum and/or the extract of the said gum are preferable.

The preferable extraction solvent is water. The aqueous extract is used as it is, or after concentration, dilution and/or purification.

The dried powder and/or the extract also can be used after purified by means of column chromatography.

The said enhancer of this invention was orally administered, for example. The daily dose of the said enhancer depends on the formulation, sexuality, age, and body weight of the patient, but usually 1 to 100 g, preferably 10 to 50 g, more preferably 10 to 20 g, and it was administered in the form of solution in water or hot water, or in the form of aqueous drink agent.

As in the case of a drink agent, if necessary, an appropriate amount of sweetener such as sugar, honey, glycerin and aspartame, spice such as garlic and ginger, perfume such as fruity flavor, antioxidant such as ethylenediamine disodium salt and sodium thiosulfate, amino acid such as leucine, methionine, lysine, and taurine, and vitamin such as vitamin A, vitamin B, vitamin C, vitamin D, vitamin E, and nicotinamide, can be added to the said drink formulation.

Natural juice such as orange juice, grapefruit juice, and vegetable juice (kale, young leaves of barley), and/or the extract of crude drug such as Ginseng and Young Deer Horn also can be added to the said drink formulation for oral administration.

### EXAMPLES

The effect of GA on a decrease of cholinesterase activity caused by an organophosphorous pesticide was tested.

### 1.Animals

Male ddY mice weighing 25-30 g provided by SHIMIZU Laboratory Supplies co., Ltd. were used. They were kept in a circumstance where temperature and humidity were automatically controlled, and feed and water were freely available.

### 2.Reagents

GA was obtained from SANKYO Foods Industry Corp. Chlorpyrifos(CP), Assay Kit for Cholinesterase(ChE), lactate dehydrogenase(LDH), glutamate oxaloacetate transaminase(GOT), and glutamate pyruvate transaminase(GPT) were obtained from Wako Pure Chemical Industries,Ltd. All other reagents were commercially available.

### 3.Method

Mice were assigned to the following 4 groups of 5 mice. each.
1) Group1(Control): saline and cotton seed oil were orally administered for 4 weeks.
2) Group2(GA): GA(1200 mg/kg/day) dissolved in saline was orally administered for 4 weeks.
3) Group3(CP): CP(8mg/kg/day/0.2 ml) dissolved in cotton seed oil was orally administered for 4 weeks.
4) Group4(CP+GA): CP(8 mg/kg/day/0.2 ml) dissolved in cotton seed oil and GA(1200 mg/kg/day) dissolved in saline were orally administered for 4 weeks.

### 4.Assay Method for Serum-Enzyme

Blood samples were taken from mice of each group 24 h after the final administration of agent under pentobarbital anesthesia. Bood samples obtained by direct cardiac puncture were centrifuged(2000×g) for 15 min at 4C to give serum sample for the assay of enzymes. Each enzymic activity(ChE, LDH, GOT, and GPT) was assayed using Assay Kit for the enzyme.

### 5.Statistical Analysis

Results were expressed as mean±SE for the group. Comparisons of parameters between different groups were evaluated by Student's t-test. Results were considered statistically significant when p<0.01.

### 6.Results

Results were shown in Table 1.

TABLE 1
Effects of CP and GA on enzymic activity in serum
Enzymic activity in serum (IU/L)

| | ChE | LDH | GOT | GPT |
|---|---|---|---|---|
| Group 1 | 432.3 ± 38.2 | 312.6 ± 29.0 | 42.8 ± 10.8 | 5.4 ± 0.3 |
| Group 2 | 407.7 ± 75.5 | 396.4 ± 24.4 | 58.6 ± 15.2 | 5.6 ± 0.8 |
| Group 3 | 9.7 ± 3.2 | 527.9 ± 35.1 | 106.9 ± 30.7 | 8.0 ± 1.2 |
| Group 4 | 79.1 ± 11.4* | 431.1 ± 22.8 | 95.8 ± 23.6 | 7.8 ± 2.4 |

*p < 0.01

Daily administration of GA gave no effect on enzymic activities in serum(Group2). Administration of CP remarkably decreased ChE activity, while slightly increased serum LDH, GOT and GPT(Group3). Combination of CP and GA significantly inhibited the decrease of ChE, but gave no effect on the increase of serum LDH, GOT or GPT.

### Industrial Applicability

Since GA specifically inhibit only the decrease of ChE Activity with no effect on LDH etc., the invention is useful as a specific medicine for preventing and/or treating only ChE activity.

1-10. (canceled)

11. A method for treating liver damage caused by inhibition of cholinesterase activity, which comprises administering a therapeutically effective amount of a water-soluble gum obtained from the stem and/or branch of Acacia species to a patient in need of the treatment.

12. The method according to claim 11, wherein the water-soluble gum is Gum Arabic.

13. A method for treating liver damage caused by an organophosphorous pesticide or an anticancer agent, which comprises administering a therapeutically effective amount of a water-soluble gum obtained from the stem and/or branch of Acacia species to a patient in need of the treatment.

14. The method according to claim 13, wherein the water-soluble gum is Gum Arabic.

15. A method for treating liver damage caused by Chlorpyrifos, which comprises administering a therapeutically effective amount of a water-soluble gum obtained from the stem and/or branch of Acacia species to a patient in need of the treatment.

16. The method according to claim 15, wherein the water-soluble gum is Gum Arabic.
